(19) United States
(12) Patent Application Publication
(10) Pub. No.: US 2023/0286830 A1
(43) Pub. Date: Sep. 14, 2023

(54) INTEGRATED WATER AND OBJECTS DISINFECTION APPARATUS

(71) Applicant: STRAUSS WATER LTD, Or Yehuda (IL)

(72) Inventors: Meital Shafir, Kiryat Ono (IL); Omer Ben Baruch, Kiryat Ono (IL); Eyal Krystal, Kfar-Saba (IL); Hella Frenkel, Kfar-Saba (IL); Orly Nahum, Ramat Gan (IL)

(73) Assignee: STRAUSS WATER LTD, Or Yehuda (IL)

(21) Appl. No.: 18/013,800

(22) PCT Filed: Jul. 1, 2021
(86) PCT No.: PCT/IL2021/050816

§ 371 Date: Dec. 29, 2022

(30) Foreign Application Priority Data
Jul. 1, 2020 (IL) 275790

(51) Int. Cl. C02F 1/02; A61L 2/07; A61L 2/26

(52) U.S. Cl. CPC C02F 1/02; A61L 2/07; A61L 2/26; C02F 2303/04; C02F 2209/02; C02F 2307/10; A61L 2202/14

(57) ABSTRACT

An integrated disinfection apparatus for disinfection of drinking water and an object comprising: a steam disinfection unit for disinfecting objects by steam; a water heat-disinfection unit for disinfecting water by heat and dispensing disinfected drinking water at a desired temperature upon user demand; and at least one water inlet feeding water from a water source to the steam disinfection unit, the water disinfection unit or both.

### TECHNOLOGICAL FIELD

The present disclosure concerns a water disinfection apparatus, specifically an apparatus which is configured to provide heat-disinfected drinking water for consumption or for various uses and to steam-disinfect various objects therein.

### BACKGROUND

Systems for disinfection/sterilization of various objects by steam are known. Typically, such systems are domestically used for sterilization of items utilized for infant care, such as feeding bottles, bottle nipples, pacifiers, feeding utensils, etc. Such systems can also be used for sterilizing various objects, such as medical devices contaminated by various microbiological contaminants. Usually, such objects' sterilization systems are stand-alone devices, which require the user to manually fill a container with water, and either turn on a heating unit for turning the water into steam or insert the apparatus into a domestic microwave apparatus for heating up the water to form water vapors.

Water dispensers are also known for on-demand dispensing of water at various temperatures.

### GENERAL DESCRIPTION

The present disclosure provides an integral disinfection apparatus which can provide both heat-disinfected drinking water and carry out steam disinfection of objects needed for such disinfection. The apparatus of this disclosure provides a compact system which meets domestic disinfection needs, for example disinfecting items utilized for infant care, and providing disinfected drinking water at a controlled and pre-determined temperature for on-demand preparation of baby feeding formulae.

It is to be noted that in the context of the present disclosure, the term disinfect (or any lingual variation thereof) means to denote a significant reduction in amount of microbiological species in the water or onto the object treated by the system described herein. The term also means to encompass sterilization of the water and/or objects treated by the system. The terms “disinfection” and “sterilization” will be used interchangeably herein, unless otherwise specifically indicated.

Thus, in an aspect thereof, the present disclosure provides a disinfection apparatus comprising a steam disinfection unit for disinfecting objects by steam, a water heat-disinfection unit for disinfection of water by heat and dispensing disinfected drinking water upon user demand, and at least one water inlet for feeding water from a water source to the steam disinfection unit, the water disinfection unit or both.

The disinfection apparatus of the present disclosure may be a stand-alone apparatus, i.e. not connected to a water grid or another device. In such cases, water can be supplied to the disinfection apparatus from a designated reservoir connectable to the water inlet and functioning as the water source, or manually fed by the user. Water can be provided separately to each of the steam disinfection unit and the water heat-disinfection unit via dedicated water inlets configured at each of the units, or from a joint water inlet.

However, in a preferred embodiment, the water inlet of the apparatus is configured to connect to a running water source (e.g. a household water supply line or water faucet), or to a water treatment apparatus from which treated water (e.g. filtered or purified water, not disinfected/sterilized) can be fed into the disinfection apparatus through a linking conduit that links the water treatment apparatus to the water inlet(s) of the disinfection apparatus.

In some embodiments, the water inlet is configured for association with a water dispenser, typically a water dispenser that comprises a water treatment and/or purification module (such as a water filter, a UV lamp, a purification cartridge, etc.), via a connection cable that permits water flow from the water dispenser to the disinfection apparatus. Thus, water from which various contaminants have been removed (referred to herein as treated water) can be fed directly from the water dispenser into the disinfection apparatus. Feeding of treated water may reduce the time required to disinfect/sterilize the water (in case some microorganisms have already been reduced from the source water by the water dispenser), minimize scale formation in the disinfection apparatus and/or prevent formation of high concentrations of metallic species in the heat-disinfected water (as the metallic species are removed by the treatment module of the water dispenser prior to introduction into the disinfection apparatus).

Feeding of treated water from the water dispenser to the disinfection apparatus can be carried out upon user demand, i.e. when a user intends to utilize the disinfection apparatus. Alternatively, the disinfection apparatus can automatically draw treated water (or be fed with water) from the water dispenser when the level of water drops below a predetermined level in the water disinfection unit and/or the steam disinfection unit of the disinfection apparatus.

Further, by an embodiment, the disinfection apparatus is configured to receive electrical current for its operation from the water dispenser, which is typically electrically operated. The electrical connection between the disinfection apparatus and the water dispenser can be via an electrical connection cable, that may be a stand-alone cable or may be part of an integral connection cable that contains both an electrical cable and said water linking conduit.

In order to permit a user to disconnect the disinfection apparatus from the water treatment apparatus when needed (e.g. for storage or cleaning), the disinfection apparatus is, by an embodiment, configured for selective fluid- and electrical-association with the water treatment apparatus.

As noted the disinfection apparatus comprises a steam disinfection unit and a water heat-disinfection unit, which are fed water for their operation, as will be described further below, through at least one water inlet, typically a mutual apparatus water inlet. The water inlet is, by one embodiment, configured to concomitantly feed water from the water source to the steam disinfection unit and the water heat-disinfection unit. In another embodiment, the water inlet is configured to selectively feed water from said water source to the steam disinfection unit or to the water heat-disinfection unit.

By some embodiments, the steam disinfection unit comprises a steam disinfection chamber that is configured to accommodate objects to be disinfected by steam therein, and a water heating chamber associated with the steam disinfection chamber. The water heating chamber comprises a water reservoir and a first heating module configured to heat the water in the water reservoir to form steam. The water heating chamber is in fluid communication with the steam disinfection chamber for permitting steam to flow from the water heating chamber into to the steam disinfection chamber, thus permitting objects accommodated within the steam disinfection chamber to be disinfected by steam.

Typically, disinfection of objects in the steam disinfection chamber is carried out by exposing the objects contained therein to steam at a defined temperature for a defined period of time. The temperature of the steam and the exposure duration can be varied upon the desired degree of disinfection, and/or according to local health regulations. For example, the objects may be exposed to steam at a temperature of at least 90° C. for a period of time of at least 6 minutes in order to obtain disinfection/sterilization by the steam.

The steam disinfection chamber and the water heating chamber may be integral with one another, for example via a mutual wall in which a steam-flow opening is defined. By an embodiment, a bottom wall of the steam disinfection chamber constitutes a top wall of the water heating chamber. By a further embodiment, the steam disinfection chamber and the water heating chamber define together a joint space within the steam disinfection unit.

The steam disinfection chamber and/or the water heating chamber may comprise one or more temperature sensors for measuring and/or indicating the temperature within the steam disinfection unit.

For safety purposes, the steam disinfection unit can comprise various safety mechanisms. For example, the steam disinfection chamber may comprise a vent and/or a pressure-release valve for venting out excess steam. The steam disinfection chamber may be made of or coated by a heat insulating material, preventing a user from being exposed to high temperatures when the steam disinfection unit is in operation.

By another embodiment, the steam disinfection chamber can comprise a removable cover or a closable access hatch for permitting access into the steam disinfection chamber (e.g. for inserting and extracting the objects to be steam disinfected/sterilized or the disinfected/sterilized objects, respectively). For safety purposes, the steam disinfection chamber may comprise a locking mechanism configured to lock the removable cover or the closable access hatch in a locked state, preventing user access into the steam disinfection chamber when the temperature in the chamber is higher than a predetermined threshold temperature. Once temperature within the steam disinfection chamber drops below this predetermined threshold temperature, the lock can be opened (or is automatically opened) to permit access into the interior of the steam disinfection chamber.

The steam disinfection chamber is configured to accommodate one or more objects to be steam disinfected/sterilized therein. By an embodiment, the steam disinfection chamber is configured to accommodate one or more object-supporting elements, for supporting one or more objects to be disinfected within the steam disinfection chamber. For example, the object-supporting element may be a basket, into which small objects (e.g. pacifiers, feeding bottle nipples, small toys, etc.) can be placed and held during the disinfection process. In another example, the object-supporting element may be designed to hold feeding bottles in a bottom-up configuration (i.e. with their opening facing downwards). The one or more object-supporting elements may be user-removable, permitting their insertion and extraction from the steam disinfection chamber when needed.

As during cooling of the steam disinfection chamber (after completion of the disinfection process) residual steam may be condensed on the disinfected/sterilized objects, the steam disinfection unit may comprises a drying and/or cooling module configured for drying and/or cooling the objects in the steam disinfection chamber after completion of disinfection. An exemplary drying module may be a hot/cold air blower or any other suitable drying module.

In some embodiments, the steam disinfection chamber may be at least partially transparent to permit a user to view the objects accommodated in the steam disinfection chamber during operation of the unit.

As noted, the apparatus further comprises a water heat-disinfection unit, that permits disinfection of source water or treated water to obtain heat disinfected/sterilized drinking water. The steam disinfection unit and the water heat-disinfection unit may be integral one with the other in order to afford for a relatively compact and streamlined apparatus. By an embodiment, the steam disinfection unit and said water heat-disinfection unit are integral one with the other along at least one joint common wall.

The apparatus may have a housing that encases the steam disinfection unit and the water heat-disinfection unit. The housing may be transparent or translucent (at least in parts thereof), or may be opaque.

The water heat-disinfection unit comprises a water heat-treatment chamber, a second heating module that is configured to change and maintain the temperature of the water within the water heat-treatment chamber according to a defined temperature profile, and a disinfected drinking water dispensing outlet for dispensing disinfected/sterilized drinking water from the water heat-treatment chamber upon user demand.

In some embodiments, the temperature profile comprises bringing the water in the water heat-treatment chamber to boil, maintaining the water in boiling temperature for a predefined time period, permitting the water to cool to a dispensing temperature, and maintaining the cooled water at said dispensing temperature. Such a temperature profile permits disinfecting/sterilizing the water in order to obtained disinfected/sterilized drinking water, while maintaining the disinfected water in the water heat-treatment chamber at a constant desired temperature after disinfection, ready for dispensing at a dispensing temperature when desired.

For example, the disinfected/sterilized drinking water may be utilized in order to prepare baby feeding formulae, and hence need to be maintained at a suitable temperature after being sterilized. In some embodiments, the dispensing temperature is in the range of between about 20° C. and about 40° C., preferably between about 25° C. and about 37° C.

In some embodiments, the water in the water heat-treatment chamber are brought to a temperature of at least 90° C. (i.e. to a temperature of between about 90 and 105° C., which can be determined and controlled according to the geographical location of the device), and maintained at such temperature for at least about 2 minutes before allowing the water to cool to the dispensing temperature (e.g. as per the Israeli Ministry of Health recommendations).

Cooling of the disinfected water in the water heat-treatment chamber may be carried out by passive cooling. Alternatively, cooling can be carried out by operation of a cooling module, e.g. cooling ribs, stirring mechanism, cooling the chamber walls, heatsink arrangement, heat pipes, gas-based cooling system, etc.

While the water disinfection unit is fed water from a water source via the apparatus inlet, the water heat-treatment chamber can further comprise a water feeding opening, permitting water feeding to the water heat-treatment chamber if desired, add various additives into the water if required, or add scale cleaning agents to permit cleaning of the water heat-treatment chamber from undesired accumulated scale.

The apparatus typically comprises a control panel for controlling operation of the apparatus. Such a control panel may include, inter alia, operation buttons, a user interface, a display panel, and/or various indicators. In some embodiments, the apparatus comprises one or more temperature indicators for indicating temperature within the water heat-treatment chamber and/or the steam disinfection chamber. For example, the progression of heating and/or cooling can be indicated by consecutive indicators, that can progressively indicate the temperature of the water (cooling and/or heating of the water). An alternative mode of indication can be a display of the actual measured temperature and/or the time remaining for heating/cooling to complete.

The steam disinfection unit and the water heat-disinfection unit may be independently operated by the user. However, it is also contemplated that operation of one of the units may be delayed or blocked by the controller during operation of the other of the units—for example for optimal electricity utilization. Alternatively, the steam disinfection unit and the water heat-disinfection unit can be operated simultaneously.

It is further contemplated that operation of the apparatus may be carried out by a water treatment apparatus (i.e. a water dispenser/bar) when the disinfection apparatus is electrically linked thereto. For this purpose, the electrical connection cable linking the disinfection apparatus to the water treatment apparatus can also comprise one or more control lines.

In addition, once connected to the water treatment apparatus, the operation of the disinfection apparatus can be controlled by the water treatment apparatus. In other words, once connected, the disinfection apparatus can be controlled and operated automatically by the water treatment apparatus, e.g. the water treatment apparatus can automatically feed treated water to the disinfection apparatus, control various parameters of its operation, provide various indications to the user on the operation of the disinfection apparatus, update programming of the disinfection apparatus, etc.

The disinfection apparatus can also be wirelessly or network operated/controlled, e.g. programmed, operated, configured to receive/transmit various data (for example utilization data, process parameters, operation/utilization statistics, etc.). The disinfection apparatus can also be configured for wireless communication with the water treatment apparatus, thus permitting the water treatment apparatus to wirelessly control operation of the disinfection apparatus.

As noted, the disinfection apparatus can be connected to a main unit, e.g. a water dispenser. However, it is also contemplated within the scope of this disclosure that the disinfection apparatus will be further linked to other types of apparatus that dispense water for various uses. It is further contemplated within the scope of this disclosure, that various types of water-utilization units be linked to the disinfection apparatus, such that the disinfection apparatus can control operation of such water-utilization units.

As used herein, the term about is meant to encompass deviation of ±10% from the specifically mentioned value of a parameter, such as temperature, pressure, concentration, etc.

Whenever a numerical range is indicated herein, it is meant to include any cited numeral (fractional or integral) within the indicated range. The phrases ranging/ranges between (or is in a range of between) a first indicate number and a second indicate number and “ranging/ranges from” a first indicate number “to” a second indicate number are used herein interchangeably and are meant to include the first and second indicated numbers and all the fractional and integral numerals therebetween.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a perspective view of a sterilization apparatus according to an embodiment of this disclosure.

FIG. 2 is a perspective view of the sterilization apparatus of FIG. 1, however, with pars of its housing made transparent to show internal elements.

FIG. 3 is a longitudinal cross section of the apparatus of FIG. 1 along line FIG. 4 is a perspective view of the sterilization apparatus of FIG. 1, however, with pars of its housing removed to show internal elements.

FIG. 5 is a back view of the sterilization apparatus of FIG. 2.

FIG. 6 is a longitudinal cross section through an apparatus according to another embodiment of this disclosure, that includes an active cooling module.

### DETAILED DESCRIPTION OF EMBODIMENTS

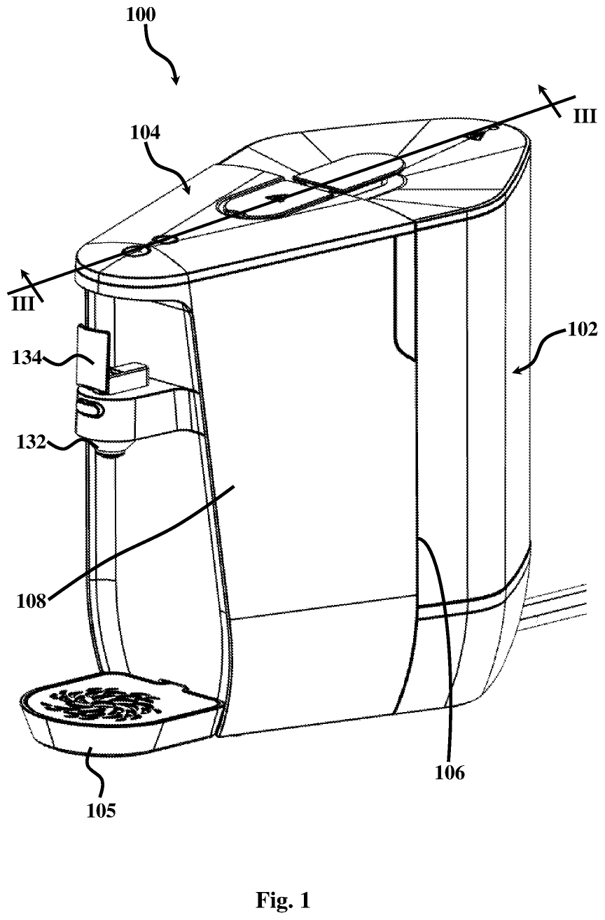

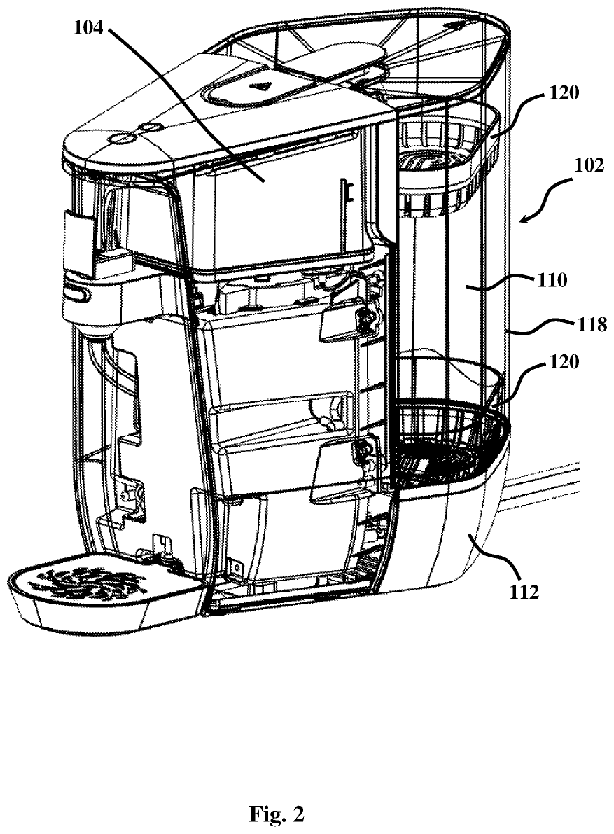

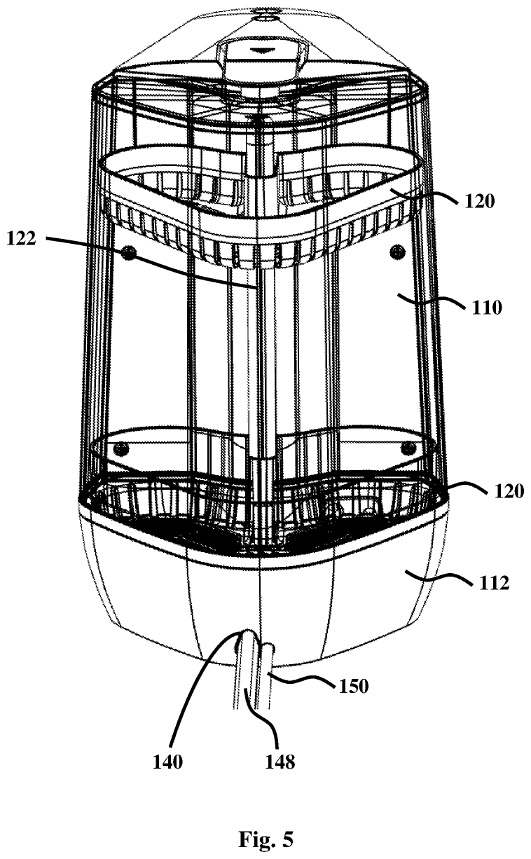

Referring to FIGS. 1-5, shown is an apparatus according to an embodiment of this disclosure. Sterilization apparatus 100 comprises a steam disinfection unit 102 and a water heat-disinfection unit 104, the structure and operation of which will be described below, and in this specific example are integral with one another along a mutual wall 106. The apparatus further has a housing 108, parts of which have been made transparent in FIGS. 2 and 5 and parts of which have been removed in FIG. 4, for ease of showing internal parts of the apparatus. Drip tray 105 is position below dispensing outlet 132, and functions to collect spillage or drops of water which may unintentionally trickle from outlet 132. Tray 105 can also serve as a frontal tilt-support to prevent unintentional tilting of the apparatus towards the user during operation of the apparatus.

With specific reference to FIGS. 2-5, steam disinfection unit 102 comprises a steam disinfection chamber 110 and a water heating chamber 112. The steam disinfection chamber 110 and the water heating chamber 112 are vertically stacked, such that the bottom wall 114 of steam disinfection chamber is the top wall of the water heating chamber. Water heating chamber 112 comprises a water reservoir 113 and a first heating module generally designated 116 for heating water fed into the water reservoir 113 in order to produce steam. One or more steam flow-permitting openings (not shown) are defined in wall 114 in order to permit steam to flow from the water heating chamber 112 to the steam disinfection chamber 110 during operation of the steam disinfection unit. However, it is also contemplated that wall 114 will be absent, resulting in formation of a unified joint disinfection volume consisting of the volume of the steam disinfection chamber and the water heating chamber and permitting free flow of steam therewithin.

In this specific example, the housing portion 118 constituting external walls of the steam disinfection chamber 110 can be removed by the user in order to gain access to the objects accommodated therein. However, it is also contemplated that the steam disinfection chamber may have a covered hatch or an opening (side or top opening), that permits access to the interior of the steam disinfection chamber. Housing portion 118 or a covered hatch can be configured with a lock (not shown) in order to permit locking of the steam disinfection chamber during operation of the steam disinfection unit and/or as long as the temperature within the steam disinfection chamber is above a predetermined threshold.

One or more object-supporting elements can be provided in the steam disinfection chamber 110 in order to support and hold the objects to be disinfected/sterilized during the steam disinfection/sterilization process. In this specific example, the object-supporting elements comprise baskets 120 attached to and vertically displaced one from the other along support pole 122. The object-supporting elements may be fixed within the steam disinfection chamber or may be removable by the user.

For safety purposes, the sterilization chamber is provided with a steam release opening 124 and/or pressure relieve valve (not shown) permitting release of excess steam from the steam disinfection chamber.

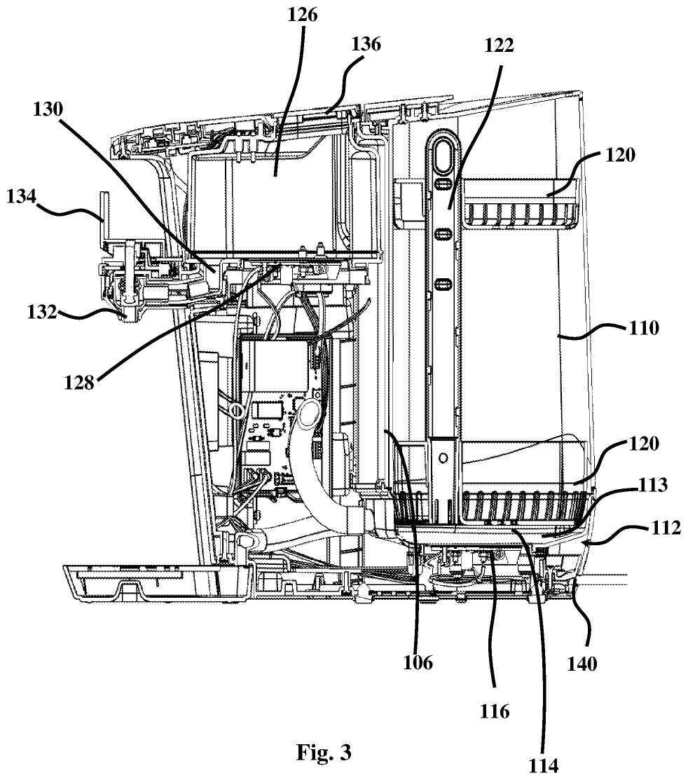

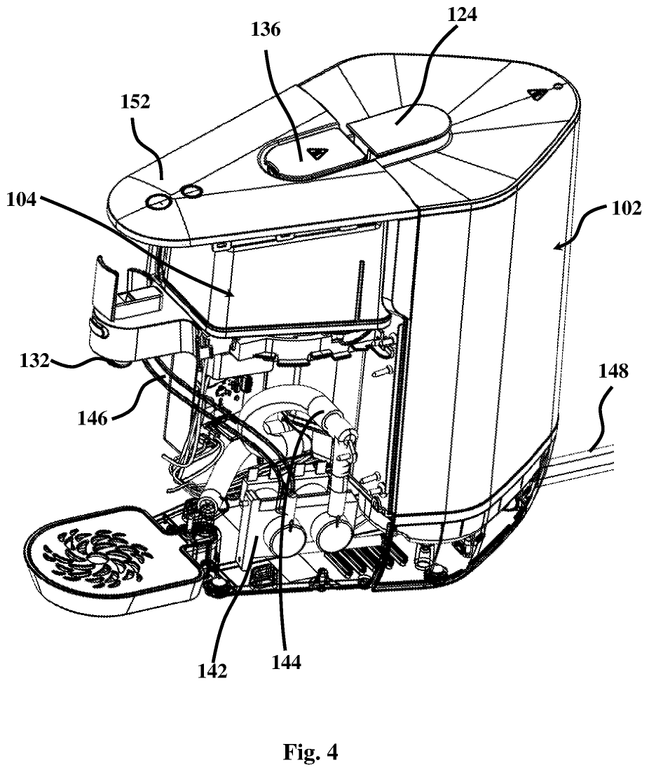

Water heat-disinfection unit 104, as best seen in FIG. 3, comprises a water heat-treatment chamber 126 and a second heating module generally designated 128 at the bottom of chamber 126. The water heat-treatment chamber has a disinfected/sterilized water outlet 130 which is in liquid communication with dispensing outlet 132, enabling dispensing of disinfected/sterilized drinking water from the water heat-treatment chamber upon user demand. In this specific example, dispensing is permitted by operation of user-operable dispensing mechanical lever 134. However, it is also contemplated that dispensing may be operated electro-mechanically.

The water heat-disinfection unit operates according to a pre-determined temperature profile or cycle of operation in order to heat-disinfect/sterilize a volume of water within the water heat-treatment chamber and maintain the water, post-disinfection, at a predetermined temperature ready for use at said predetermined temperature (i.e. a temperature suitable for baby feeding formulae). Hence, during a single cycle of operation, the second heating module 128 is operated to bring the water within the water heat-treatment chamber 126 to boil (e.g. 90-105° C.), maintain the water in boiling temperature for a predefined time period (e.g. for at least 2 minutes), permit the water to cool to a dispensing temperature (e.g. 30-40° C., either passively or actively), and maintain the cooled water at said dispensing temperature. Release of steam from the water heat-treatment chamber during boiling of water is enabled by steam-release valve (or hatch) 136.

Water from a water source is fed into the disinfection apparatus via water inlet 140. Water inlet 140 is in liquid communication with manifold 142, which can control distribution of water into water reservoir 113 and water heat-treatment chamber 126 via tubings 144 and 146, respectively. Hence water can be fed concomitantly or independently into water reservoir 113 and water heat-treatment chamber 126.

Water inlet 140 can be linked to a water source, such as a source water reservoir or a water supply line, or can be connected, as noted above, to a main water treatment apparatus such as a main water dispenser (not shown). Such connection is permitted by water conduit 148, which permits water flow from the main water dispenser to the disinfection apparatus. In case the disinfection apparatus is also electrically linked to the main water dispenser, such electrical communication is established via electrical cable 150 (best seen in FIG. 5). Water conduit 148 or electrical cable 150 may also be integrated with a control and communication line and for allowing the disinfection apparatus to be controlled by the main water dispenser or to provide various indications to the main water dispenser. Thus, control of the disinfection apparatus may be permitted via control panel 152 and/or via the main water dispenser (when the disinfection apparatus is electrically/controllably linked to such a main water dispenser).

Treated water can be fed from the main water dispenser to the disinfection apparatus upon demand of water initiated by the disinfection apparatus, or automatically when a level of water drops below a threshold level in either or both of the water reservoir 113 and the water heat-treatment chamber 126.

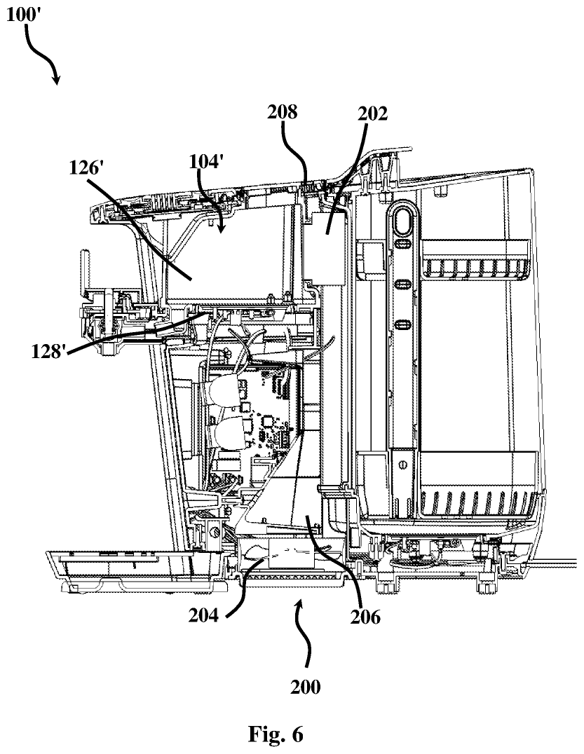

An exemplary sterilization apparatus 100′ that includes an active cooling module is shown in FIG. 6. In order to permit controlled and faster cooling of water heat-disinfection unit 104′, the apparatus includes a cooling module, generally designated 200.

Cooling module 200 includes, in this example, a heat sink 202 that is in content with the water inside water heat-treatment chamber 126′. Heat sink 202 is typically made of one or more metals. Contact of the heat sink 202 with the heated water, causes transfer of heat from the water to the heat sink, thereby actively cooling the water within heat-treatment chamber 126′. In order to remove heat from the heat sink, a rotatable fan 204 is located at the bottom of apparatus 100′ and is configured to introduce ambient or cooled air into the apparatus. An air funneling element 206 is located above the fan 204 and extends vertically to permit forced flow of air towards the heat sink 202 to remove heat therefrom, as well as to remove heat from the second heating module generally designated 128′ at the bottom of chamber 126′. Venting ports 208 are located at the top of the apparatus, enabling release of hot air from the apparatus.

1. A disinfection apparatus comprising:
a steam disinfection unit for disinfecting objects by steam;
a water heat-disinfection unit for disinfecting water by heat and dispensing disinfected drinking water at a desired temperature upon user demand; and
at least one water inlet feeding water from a water source to the steam disinfection unit, the water disinfection unit or both.

2. The apparatus of claim 1, wherein said water inlet is configured to concomitantly feed water from said water source to the steam disinfection unit and the water heat-disinfection unit.

3. The apparatus of claim 1, wherein said water inlet is configured to selectively feed water from said water source to the steam disinfection unit or to the water heat-disinfection unit.

4. The apparatus of claim 1, wherein the steam disinfection unit comprises:
a steam disinfection chamber configured to accommodate objects to be disinfected by steam; and
a water heating chamber comprising a water reservoir and a first heating module configured to heat the water in the water reservoir to form steam, the water heating chamber being in fluid association with said steam disinfection chamber for permitting steam to flow from the water heating chamber into to the steam disinfection chamber.

5. The apparatus of claim 4, wherein the steam disinfection chamber and the water heating chamber are integral with one another.

6. The apparatus of claim 5, wherein the steam disinfection chamber and the water heating chamber define together a joint space within the steam disinfection unit.

7. The apparatus of claim 5, wherein a bottom wall of the steam disinfection chamber constitutes a top wall of the water heating chamber.

8. (canceled)

9. (canceled)

10. The apparatus of claim 4, wherein the steam disinfection chamber comprises a removable cover or a closable access hatch for permitting access into the disinfection chamber.

11. (canceled)

12. The apparatus of claim 4, wherein the steam disinfection chamber is configured to accommodate one or more object-supporting elements, for supporting one or more objects to be steam-disinfected within the steam disinfection chamber.

13. (canceled)

14. The apparatus of claim 4, wherein the steam disinfection unit comprises a drying and/or cooling module configured for drying and/or cooling the objects in the steam disinfection chamber after completion of disinfection.

15. The apparatus of claim 1, wherein said water heat-disinfection unit comprises:
a water heat-treatment chamber,
a second heating module configured to change and maintain the temperature of the water within the water heat-treatment chamber according to a defined temperature profile, and
a disinfected drinking water dispensing outlet for dispensing disinfected drinking water from the water heat-treatment chamber upon user demand.

16. The apparatus of claim 15, wherein said temperature profile comprises bringing the water to boil, maintaining the water in boiling temperature for a predefined time period, permitting the water to cool to a dispensing temperature, and maintaining the cooled water at said dispensing temperature.

17. The apparatus of claim 16, wherein said dispensing temperature is in the range of between about 20° C. and about 40° C., and the water in the water heat-treatment chamber are brought to a temperature of at least 90° C. and maintained at such a temperature for at least about 2 minutes before allowing the water to cool to the dispensing temperature.

18. (canceled)

19. The apparatus of claim 15, wherein the water heat-treatment chamber further comprises a feeding opening, permitting water feeding and/or additives feeding to the water heat-treatment chamber.

20. The apparatus of claim 15, further comprising a water cooling module for cooling disinfected drinking water after heat-disinfection treatment in the water heat-treatment chamber.

21. (canceled)

22. The apparatus of claim 1, comprising one or more temperature indicators for indicating temperature within the water heat-treatment chamber and/or the steam disinfection chamber.

23. (canceled)

24. The apparatus of claim 1, wherein said steam disinfection unit and said water heat-disinfection unit are integral one with the other.

25. The apparatus of claim 1, wherein said steam disinfection unit and said water heat-disinfection unit are integral one with the other a joined along a common wall.

26. The apparatus of claim 1, wherein said water inlet is configured for feeding water from a running water source.

27. The apparatus of claim 1, wherein said water inlet is configured for feeding treated water from a water treatment apparatus into the disinfection apparatus.

28-32. (canceled)
